(19) United States
(12) Patent Application Publication
(10) Pub. No.: US 2026/0219732 A1
(43) Pub. Date: Jul. 30, 2026

(54) A DEVICE FOR TRACKING USER EYE AND HEAD POSITION

(71) Applicant: Shaurya Khosla, Chandigarh (IN)

(72) Inventor: Shaurya Khosla, Chandigarh (IN)

(21) Appl. No.: 19/144,571

(22) PCT Filed: Dec. 28, 2023
(86) PCT No.: PCT/IN2023/051241

§ 371 Date: Jun. 30, 2025

(30) Foreign Application Priority Data
Dec. 28, 2022 (IN) 202211061536

(51) Int. Cl. G06F 3/01; A61B 5/00; G06V 30/242; G09B 17/00; G09B 17/02; G10L 15/08

(52) U.S. Cl. CPC G06F 3/015; A61B 5/746; G06F 3/012; G06V 30/242; G09B 17/003; G09B 17/02; G10L 15/08; G10L 2015/088

(57) ABSTRACT

A device (10) for tracking user eye and head position. The device, configured to be attached to an optical wear (1), includes a controller (38), gyroscopes (32), and an alert unit (40). The gyroscopes (32) transmit input signals corresponding to the head inclination angle to the controller (38), wherein the controller (38) is configured to alert the user if the inclination angle of the head is more than a predefined threshold. Moreover, the device (10) can also be configured to track user eye positions comprises one or more EEG sensors (86) additionally, wherein the controller (88) is configured to compare brain waves signals captured by the one or more EEG sensors (86) with a reference data to track the position of the user's eyes (84). The reference data is being generated by capturing the brain waves of a user while the user is instructed to scan an EEG calibration sheet (80) from top to bottom, during a device's calibration process.

### FIELD OF THE DISCLOSURE

The present disclosure generally relates to a device for tracking user eye and head position. More particularly, the present disclosure relates to a device for monitoring positioning of head of a user and generating one or more corresponding alerts. The device also tracks eye movement using EEG sensors, which can be beneficial in various applications.

### BACKGROUND OF THE DISCLOSURE

Many teenagers spend ample time studying for hours or doing their work by slouching too much, causing them to read from close unintentionally or working in poor lighting, which can weaken their vision over time by straining it without realizing that they are damaging their eyes and are more likely to develop myopia, or nearsightedness. This increases their eye number and ultimately causes them to wear optical wear. Since the number continues to grow over time, people can take some precautions, who are wearing optical wear to control/slow down their eye number.

Further, myopia is a common vision problem that often begins between the ages of 6 and 15 and progresses until about 20, affecting about 30-40% of the population. Myopia in young children is generally caused by reading/viewing habits, which has dramatically increased especially during COVID pandemic. The screen time of students having full studies being done on laptops, tablets, mobiles by staring at the screen or books for extended times the eye numbers tend to rise rapidly, as young children are not aware or do not remember in their day-to-day activities to be careful on these parameters.

After disclosures related to bifocal glasses, subsequent developments have ranged from recognizing the eye number to various types of frames designed for function and style to advanced technologies to accurately identify the number of the eye and other eye-related complications and sophisticated imaging systems already exist in the market.

Additionally, tracking eye movement presents its own set of challenges. Existing devices often struggle to create accurate eye movement, which can limit their effectiveness. There are also difficulties in implementing this technology in a manner that is comfortable and convenient for the user. For instance, some devices may be cumbersome or intrusive, which can discourage regular use.

Given these issues, there is a clear need for a more effective solution for monitoring the position of a user's head and tracking eye movement. A device that can accurately monitor the user's head position, generate alerts based on this position, create accurate electrooculograms signals for eye movement tracking would address many of the current challenges in this field.

### OBJECT OF THE DISCLOSURE

It is an object of the present disclosure to ameliorate limitations of the existing prior art by providing a device that monitors the position of a head of a user.

An object of the present disclosure is to provide a device which automatically monitors a position of head of a user.

Another object of the present disclosure is to provide a device which generates one or more alerts for an unwanted position of head of a user.

Yet another object of the present disclosure is to provide a device which generates one or more alerts in real time.

Yet another object of the present disclosure is to provide a device which measures inclination of head of a user while the device is being used.

Yet another object of the present disclosure is to provide a device which measures distance of the head of a user from an object being used.

Yet another object of the present disclosure is to provide a device which measures light intensity of light falling on an object while being used.

Yet another object of the present disclosure is to provide a device which alerts a user so as to prevent damages to the eyes of the user.

Yet another object of the present disclosure is to provide a calibration method using an EEG calibration sheet. Users begin by focusing on a grid of dots on the EEG calibration sheet. This step calibrates the eye tracking system by measuring brainwave responses as the user looks at different dots. This is crucial for applications requiring eye movement tracking, such as augmented reality glasses.

### SUMMARY OF THE DISCLOSURE

In the present disclosure, a device for monitoring for tracking user eye and head position is disclosed. The device comprises a body attached to an optical wear, a controller, one or more sensors, an alert unit, and a power unit. The controller includes a memory and a processor. The one or more sensors communicatively coupled to the controller to transmit the one or more input signals to the controller, wherein the controller is configured to compare the one or more input signals with corresponding one or more predefined threshold values and generate an output signal. The alert unit is configured to generate an alert signal based on the output signal received from the controller. Further, the one or more sensors comprises a gyroscope, wherein the gyroscope is configured to measure an angle of inclination of the user's head with respect to X, Y, and Z planes and the one or more predefined threshold values comprises a threshold angle of inclination of the user's head.

Further, the device may also include a light sensor that checks sufficient illumination on an object in the user's field of view and an illumination source to provide sufficient illumination on an object. Furthermore, the device includes a distance sensor configured to measure the distance from the device to the object. The distance sensor may comprise a lidar sensor. The one or more predefined threshold values are stored inside the memory that may be defined by the user and comprises a predefined distance between the user's head and the object, and a predefined illumination on the object. Further, the device includes an illumination source positioned to face the user's optic nerve. The illumination source provides a blue light and a red light to stimulate the optic nerve of the user to improve eyesight. The alert unit may comprise a buzzer, an alarm, or the like. The power unit may comprise a rechargeable battery.

In the present disclosure, the device is configured to track an eye position of a user. One or more EEG sensors generate an input signal in real time. The controller compares the input signal received from the one or more EEG sensors with the reference data corresponding to an electrical activity in the user's brain to track the position of the user's eyes. Further, the process of generating reference data comprises reading the brainwave response data generated from the one or more EEG sensors disposed on a forehead and a mastoid bone of the user to measure the electrical activity in the user's brain while user is to scan an EEG calibration sheet from top to bottom, during a calibration process. The measured brainwave response data is stored in the memory as the reference data.

Further, the present disclosure additionally designed to project a movable laser light, word by word on a reading material positioned in front of a dyslexic children to aid in reading, further comprises one or more cameras, a movable laser light source and an audio sensor. The one or more cameras are coupled to the controller, wherein the one or more cameras are configured to capture an image of the reading material. The captured image is transmitted to the controller to perform an optical character recognition process on the image to extract the words written on a reading material. The audio sensor coupled to the controller, wherein the audio sensor is configured to capture an audio signal corresponding to the word pronounced by the dyslexic children. The controller processes the audio signal to locate the pronounced word in the reading material and instruct the movable laser light source to move and project light to a next word on the reading material. Further, the present disclosure the controller generates an output signal corresponding to the user's mental state and an indicating unit coupled to the controller, wherein the indicating unit receive the output signal from the controller to generate an alert signal regarding the user's mental state.

The foregoing paragraphs have been provided by way of general introduction and are not intended to limit the scope of the following claims. The described embodiments, together with further advantages, will be best understood by reference to the following detailed description taken in conjunction with the accompanying drawings.

### BRIEF DESCRIPTION OF ACCOMPANYING DRAWING

The above and still further features and advantages of the present disclosure will become apparent upon consideration of the following detailed description of embodiments thereof, especially when taken in conjunction with the accompanying drawings, and wherein:

FIG. 1 illustrates an exemplary back perspective view of a device for monitoring position of head of a user in accordance with the present disclosure;

FIG. 2 illustrates an exemplary front perspective view of a device for monitoring position of head of a user in accordance with the present disclosure;

FIGS. 3-5 illustrates exemplary perspective views of a sub-device and a connector in accordance with an embodiment of the present disclosure;

FIG. 6 illustrates an exemplary view of a charging port of a device in accordance with an embodiment of the present disclosure;

FIG. 7 illustrates a schematic diagram of a device comprising one or more sensors, controller, alert unit and power unit in accordance with an embodiment of the present disclosure;

FIG. 8 illustrates a schematic diagram of a device's calibration for tracking the user's eye position in accordance with an embodiment of the present disclosure; and

FIG. 9 illustrates a schematic diagram of a device designed to aid dyslexic children in reading in accordance with an embodiment of the present disclosure.

### LIST OF REFERENCE NUMERALS

1—Optical wear
2—Temple
10—Device
12—Connector
14—Circular Attachment
16—Horizontal Bar
18—Slit
20—Sub-Device
22—Protrusion
24—Charging Port
30—Body
32—Gyroscope
34—Light sensor
36—Distance sensor
38—Controller
40—Alert Unit
42—Power Unit
80—EEG calibration sheet
82—User
84—User's eyes
86—EEG sensors
88—Controller
90—Device
92—One or more cameras
94—Movable laser light source
96—Audio sensor
98—Indicating unit

### DETAILED DESCRIPTION

Various embodiments of the present disclosure provide a device for monitoring the position of head of a user. The following description provides specific details of certain embodiments of the disclosure illustrated in the drawings to provide a thorough understanding of those embodiments. It should be recognized, however, that the present disclosure can be reflected in additional embodiments and the disclosure may be practiced without some of the details in the following description.

The various embodiments including the exemplary embodiments are now described more fully with reference to the accompanying drawings, in which the various embodiments of the disclosure are shown. The disclosure may, however, be embodied in different forms and should not be construed as limited to the embodiments set forth herein. Rather, these embodiments are provided so that this disclosure is thorough and complete, and fully conveys the scope of the disclosure to those skilled in the art. In the drawings, the sizes of components may be exaggerated for clarity.

The present disclosure is to be considered as an exemplification of the disclosure and is not intended to limit the disclosure to the specific embodiments illustrated by the figures or description below. As used throughout this application, the word “may” and “can” are used in a permissive sense (i.e., meaning having the potential to), rather than the mandatory sense (i.e., meaning must). Similarly, the words “include”, “including”, and “includes” mean including but not limited to.

The foregoing discussion of the present disclosure has been presented for purposes of illustration and description. It is not intended to limit the present disclosure to the form or forms disclosed herein. In the foregoing Detailed Description, for example, various features of the present disclosure are grouped together in one or more embodiments, configurations, or aspects for the purpose of streamlining the disclosure. The features of the embodiments, configurations, or aspects may be combined in alternative embodiments, configurations, or aspects other than those discussed above. This method of disclosure is not to be interpreted as reflecting an intention the present disclosure requires more features than are expressly recited in each claim.

Moreover, though the description of the present disclosure has included description of one or more embodiments, configurations, or aspects and certain variations and modifications, other variations, combinations, and modifications are within the scope of the present disclosure, e.g., as may be within the skill and knowledge of those in the art, after understanding the present disclosure, it is intended to obtain rights which include alternative embodiments, configurations, or aspects to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

The disclosure provides a device for monitoring the head position of a user. The device comprises a body attached to an optical wear, a controller with a memory and a processor, one or more sensors, an alert unit, and a power unit. The device aims to provide a comprehensive solution to monitor and track the user's head position with high precision. The sensors are provided to generate an input signal measuring the position of the user's head, which is then transmitted to the controller. The controller compares the input signal with one or more predefined threshold values and generates an output signal. The alert unit, receiving the output signal from the controller, generates an alert signal. The power unit provides the necessary power for the device to function.

The device includes a gyroscope as part of the sensor system, which measures the angle of inclination of the user's head with respect to the X, Y, and Z planes. This feature enhances the device's ability to accurately monitor the user's head position in three-dimensional space. The gyroscope provides a reliable and precise measurement of head position, improving the device's overall performance.

The device may also incorporate a light sensor to check sufficient illumination on an object and an illumination source to provide sufficient light. The light sensor and illumination source work in conjunction to ensure optimal lighting conditions for the device to function effectively. This feature is particularly useful in low-light conditions, ensuring the device's performance is not compromised.

The device may also include a distance sensor, such as a lidar sensor, to measure the distance from the device to the object. The lidar sensor uses light in the form of a pulsed laser to measure variable distances. This feature enhances the device's ability to accurately monitor the user's head position relative to the object, providing a more comprehensive understanding of the user's movements.

The one or more predefined threshold values are stored inside the memory by the user. The threshold value can include a threshold angle of inclination of the user's head, a predefined distance between the user's head and the object, and a predefined illumination on the object. This feature allows for a personalized and adaptable system that can be tailored to the user's specific needs and preferences.

The controller transmits the output signal to the alert unit upon detecting the input signal received from the sensors is more than the predefined threshold value. This feature ensures timely and accurate alert signals, enhancing the device's effectiveness and user-friendliness.

The device may also include an illumination source positioned to face the user's optic nerve. The illumination source can provide a blue light and a red light to stimulate the optic nerve of the user to improve eyesight. This feature adds a therapeutic aspect to the device, providing potential benefits to the user's visual health.

The alert unit of the device may comprise a buzzer, an alarm, or similar alerting mechanisms. This feature provides a variety of alert options to cater to different user preferences and needs, enhancing the device's usability and accessibility.

The power unit of the device may include a rechargeable battery. This feature ensures the device's sustainability and cost-effectiveness, reducing the need for constant battery replacements and contributing to environmental conservation.

The present disclosure relates to a device for tracking an eye position of a user. The device comprises one or more EEG sensors configured to generate one or more input signals. A controller, which includes a memory and a processor, is also part of the device. The memory stores reference data corresponding to an electrical activity in the user's brain. The controller compares the input signals received from the EEG sensors with the reference data to track the position of the user's eyes. This innovative device has the potential to revolutionize eye tracking technology, with potential applications in various fields such as psychology, neuroscience, and marketing.

The process of generating reference data involves reading the brainwave response data generated from the EEG sensors. These sensors are disposed on a forehead and a mastoid bone of the user to measure the electrical activity in the user's brain while the user scans an EEG calibration sheet from top to bottom. The measured brainwave response data is then stored in the memory as the reference data. This method ensures accuracy and reliability in the tracking of the user's eye position.

The EEG calibration sheet used in the process features a grid of dots or alphabets. This specific design facilitates the calibration process and enhances the accuracy of the reference data generated. The calibration sheet could be made of various materials and could be presented in different sizes and formats, depending on the specific needs of the user or the application.

The device is also designed to project a movable laser light, word by word on a reading material positioned in front of dyslexic children to aid in reading. It further comprises one or more cameras, which are coupled to the controller. The cameras are configured to capture an image of the reading material, which is then transmitted to the controller. The controller performs an optical character recognition process on the image to extract the words written on the reading material.

The device includes a movable laser light source and an audio sensor coupled to the controller. The audio sensor is configured to capture an audio signal corresponding to the word pronounced by the dyslexic children. The controller processes the audio signal to locate the pronounced word in the reading material and instructs the movable laser light source to move and project light to a next word on the reading material. This feature of the device aids in enhancing the reading skills of dyslexic children, thereby improving their overall learning experience.

In another embodiment, the controller generates an output signal corresponding to the user's mental state. An indicating unit is coupled to the controller, which receives the output signal from the controller to generate an alert signal regarding the user's mental state. This feature could be used in various applications such as neurofeedback therapy, meditation, and cognitive training, providing valuable insights into the user's mental state and facilitating personalized interventions.

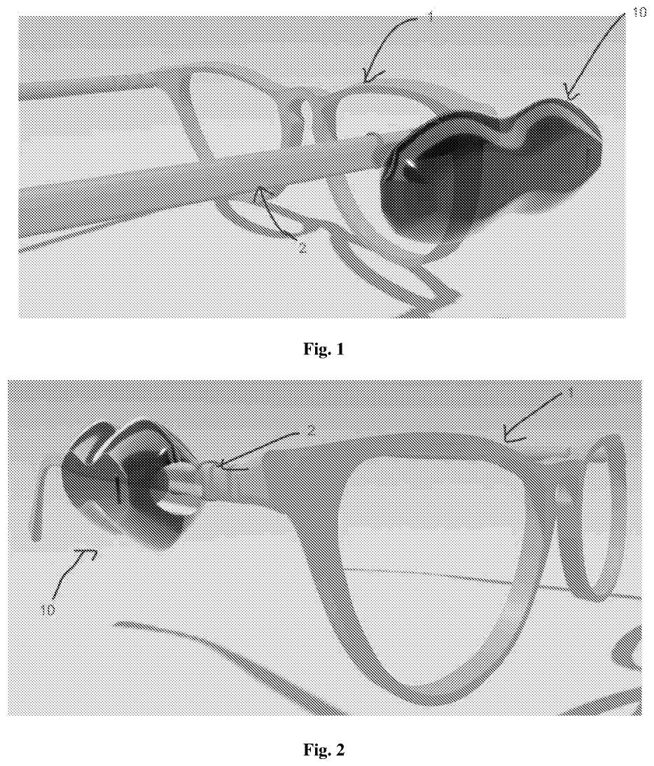

Referring to FIG. 1, the depicted device 10 is designed to monitor the position of a user's head. The device is comprised of a body that is configured to be attached to an optical wear. The optical wear could be any eye wearable device that is positioned in front of the user's eyes, such as glasses or goggles. The present disclosure relates to a device 10 for monitoring a position of head of a user. The device 10 may be configured to be used with optical wear 1 of the user as illustrated in FIGS. 1-2. In other words, the device 10 may be attached to the optical wear 1 of the user for monitoring position of the head of the user during different activities, such as reading, writing, drawing, and the like. The device 10 may measure a position of the head of the user with respect to an object the user is working on. Some non-limiting examples of the objects are a notebook, a paper, a textbook, a tablet, a computer, a laptop, and the like. The device may also be used by users involved in near sight work such as weaving, assembly of electronic components, and the like to maintain position head of user with respect to an object they are working on. Such maintained position prevents nearsightedness and other diseases that may occur on a long term.

The device 10 may comprise a sub-device 20 and a connector 12. The connector 12 may be configured to attach the device 10 to optical wear 1 of a user. In some embodiment, the device 10 may comprise a connector 12 attached therewith for connection with any one of temples 2 of the optical wear 1. In such embodiments, the connector 12 may have one or more C-shaped attachment that snap-fits with a temple 2 of the optical wear 1. In another embodiment, the connector 12 may have a circular attachment for attachment with the temple 2. In some embodiments, the device may include various connecting mechanisms such as surface twist lock in place, push in place, adhesive based connectors, magnetic connectors, and the like. In yet another embodiment, the device may be embedded in a frame of the optical wear of the user.

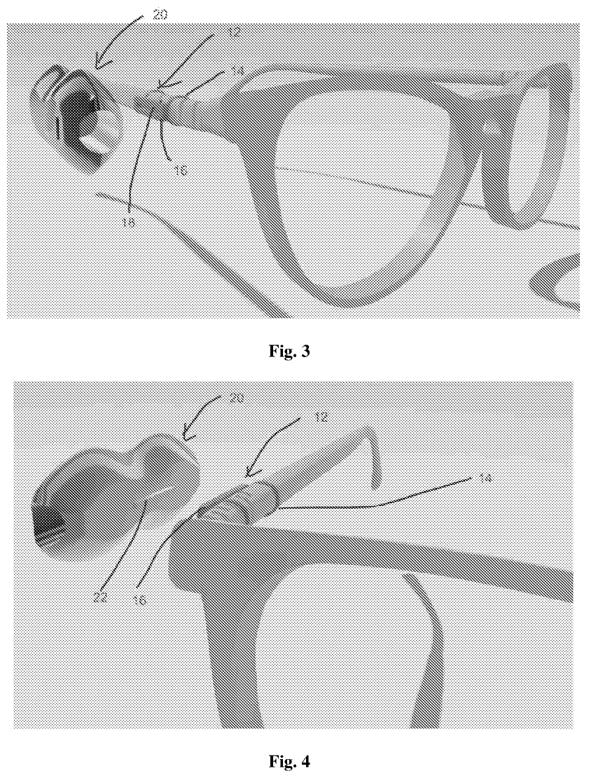

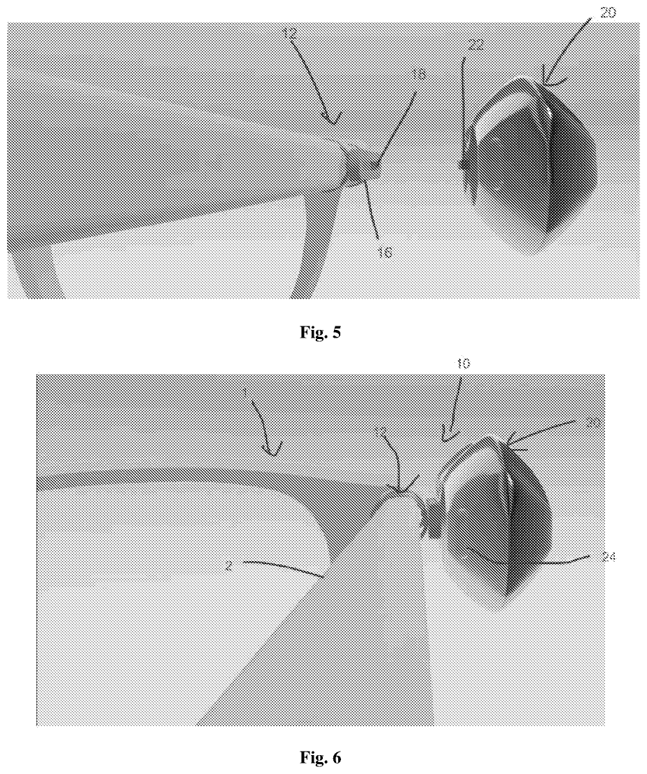

In other embodiments, the connector 12 may be separate from the sub-device 20 as illustrated in FIGS. 3-5 and may comprise circular attachment 14 at one side for attachment with any one of temples 2 of the optical wear 1. The connector 12, in such embodiment, may comprise a provision on another side for attachment with the sub-device 20. The provision may comprise a horizontal bar 16 having a slit 18 therewithin to receive the sub-device 20. The sub-device 20 may comprise a protrusion 22 at one side for attachment with the connector 12. More particularly, the protrusion 22 may be designed in a way so as to be received in the slit 18 of the provision on the connector 12 for attachment therewith.

The sub-device 20 may include one or more sensors (not shown) for measuring a position of head of the user. The device also includes a controller (not shown in fig), which comprises a memory and a processor. The controller is designed to process and store data received from the various components of the device. The controller is also configured to compare input signals with predefined threshold values and generate an output signal based on this comparison. In an embodiment, the sub-device 20 may include a gyroscope for measuring an angle of inclination of the head of the user with respect to user's body. The gyroscope is configured to generate an input signal that measures the position of the user's head. The gyroscope is communicatively coupled to the controller, allowing them to transmit the input signal to the controller for processing. In an embodiment, the gyroscope may be used to detect the angle of tilt or inclination along Y axes. The user can define a threshold angle by specifying a range. A greater value of the range allows the user to obtain more time to bend; similarly, a smaller value of the range implies the user gets less time to bend. The axis has may be mapped from 90-180 degrees. A mean position of the head may be kept at 90 degrees, so that the angle of inclination may be measured from Y-axis. The sub-device 20 may calibrate a movement of the head from its initial position to an end position by verifying displacement therebetween. For example, the user may specify the threshold angle of degree 120, so that an inclination of the head between 90 degrees and 120 degrees may be considered as a threshold limit. In an embodiment, the user may specify such angle as per their writing or reading practice, thereby making the device adaptable to user practice. In other embodiments, the gyroscope may be configured to measure both tilt/bending in forward or backward direction and sway (i.e., either side leaning) of the head of the user in X, Y, and Z planes. In such embodiments, the user can specify a threshold value for each of the tilt/bend and the sway of the head.

In some embodiments, the sub-device 20 may further comprise a light sensor. The light sensor to check sufficient illumination on an object and an illumination source to provide sufficient light. The light sensor and illumination source work in conjunction to ensure optimal lighting conditions for the device to function effectively. This feature is particularly useful in low-light conditions, ensuring the device's performance is not compromised. The light sensor checks two conditions of the user. The first condition is when a user is reading, writing, or using mobile device in poor lighting, and the second condition is if there is sufficient illumination on the object, but a brightness of the mobile device is too high. The light sensor may be activated in a certain distance range, for instance, when the object is 50 cm away from the user. Otherwise, the light sensor remains inactive in regular situations, such as sitting in the sun or strolling at night. There is a buffer time which can be set from a predefined limit for the device 10 to generate an alert. In an embodiment, the predefined limit is 10-30 seconds. In condition where none of the above two conditions are detected, the light sensor may generate a first pulse signal. The light sensor also caters to checking the time the user goes outside in the sun and stays inside by giving detailed analysis during a week.

The sub-device 20 may further include a distance sensor, e.g., a lidar sensor. The distance sensor configured to measure the distance from the device to the object. The one or more predefined threshold values are stored inside the memory by the user and comprises a threshold angle of inclination of the user's head, a predefined distance between the user's head and the object, and a predefined illumination on the object. The lidar sensor may be located in front of the device 10 to check a distance between the optical wear 1 to the object. A predefined distance is fixed for generation of a second alert and cannot be changed, as it is advised that the maximum distance between an object and the eyes of the user should be between 25-30cm for healthy eyesight.

In an embodiment, the light sensor and the lidar sensor may be located at the device's opening. The lidar sensor checks the distance from the optical wear 1 to the object. If the user bends and comes closer to the object by crossing the predefined distance, an alert is generated indicating the same and is transmitted to the user.

In an embodiment, the device 10 may include one or more processors. The one or more processors may be communicatively coupled to the one or more sensors, such as the gyroscope, the light sensor, the lidar sensor, and the like. The one or more processors may receive one or more signals generated by the one or more sensors and generate corresponding one or more alert signals.

The device 10 may include at least one alert unit. Some non-limiting examples of the at least one alert unit are a vibration unit, an audio indicator, a visual indicator, and a combination thereof. The generated one or more alert signals may be transmitted to the at least one alert unit so as to indicated generated alert in real time. The generated alert may alert the user regarding an unwanted condition. Some unwanted conditions may include reduced distance between the eyes (head) of the user and the object, less intensity of light on the object, high brightness of light of the object, and the like. For instance, the device 10 may vibrate if a current detected angle of the head exceeds the threshold angle. The vibration stop automatically if the current detected angle when the user changes the inclination of the head and the changed angle of the head is less than the threshold angle. There is a buffer time of 10 seconds which can be changed between 10-30 seconds, which means that if a user turns their head down and back up within the time limit, no alert is generated. However, if the user constantly bends their neck for more than a predefined time, for instance, 10 seconds, the device 10 may begin vibrating if the angle passes the threshold angle.

In an embodiment, the device 10 may be programmed using the one or more processors. When the device 10 detects the user is bending head for a specific time and a specific angle, which is greater than the predefined time and the threshold angle, respectively, an alert is generated, reminding the user to change position of the head in real time, for instance, to straighten up. Hence, the device 10 of the present disclosure prevents any unwanted head gestures made by the user while doing specific activities, such as reading, writing, using mobile device 10, and the like. In an embodiment, the generated at least one alert may be indicated periodically on the at least one alert unit unless a correction is made in the detected position (the distance and the inclination) of the head of the user. In another embodiment, the generated at least one alert may be indicated only once on the at least one alert unit. In yet another embodiment, the generated at least one alert may be indicated continuously on the at least one alert unit.

In some embodiments, the sub-device 20 may further include a touch portion having a capacitive touch sensor. The touch portion may be used, or touched by the user, to stop one or more alert generated by the device 10. In case, the user wants the at least one alert generating unit to be disabled for a certain amount of time, the user may postpone the indication of generated alerts for a desired time, for instance 5 minutes, by long pressing the touch portion. Such feature provides more control to the user for operating the device 10, thereby making the device 10 user-friendly.

The device 10 may include a battery for providing power to the one or more sensors, one or more processors, and the at least one alert unit. For charging of the battery, the device 10 may include a charging port 24. FIG. 6 illustrates an exemplary charging port 24 of the device 10. The device 10 may further include a charging indicator (not seen) indicating a remaining battery in the device 10. The charging indicator may be a visual indicator such as an LED. In such an embodiment, when the remaining battery is low, the LED blinks, indicating a low battery status. Other types of charging indicators known to a skilled person may also be used. In certain embodiments, an exemplary charger may have a receiving port corresponding to the charging port 24 of the device 10. In the event of the low battery, the sub device 10 may be detached from the connector 12 or the temple 2 of the optical wear 1, as the case may be, and attached to the receiving port of the charger via the charging port 24. Once the battery is fully charged, the sub-device 20 may be attached to the connector 12 or the temple 2 of the optical wear 1. Such feature makes the device 10 portable and easy to use at different locations as required. In alternate embodiment, the device may have contactless charging. In such embodiment, the device may be charged wirelessly.

In some embodiments, the device 10 may be connected to one or more mobile devices using an application. In such embodiments, a software application may be designed to control one or more functionalities of the device 10. For instance, a threshold angle for head inclination may be set using the application. Further, the generated one or more alerts may be stopped using the application. The buffer time for the user to turn their head down and back up may be adjusted by the application. The application may be installed in a user device 10. In an embodiment, the user device 10 may be connected to a server using a communication network. Different parameters selected or entered by the user through the user device may be transmitted to the device 10 via the server. In another embodiment, the user device may be connected to the sub-device 20 using near-field communication, or Bluetooth. In such embodiment, values of such parameters may be received by the one or more processors of the device 10 so as to control functioning of different parts such as the one or more sensors or the at least one alert unit.

It is submitted that even though the use of the device 10 throughout the description is illustrated with optical wear 1 of the user, the device 10 may be used with other devices as well. For instance, the device 10 may be attached to headbands, antiglare glasses, virtual reality (VR) glasses, headsets, or the like. Variations of this disclosure can be integrated into VR headsets and data collected can be used to design VR headsets and software to minimize eye and posture problems, including jerks to neck while playing games to prevent long term consequences to the health of the user. These can also effectively be programmed to hard-stop the VR activity in case of seizure, etc. Requirements that have hand-eye coordination where head angle can be accounted for through gyroscopic movement, which can be combined with other tracking systems worn on the wrist etc., in sports activities or activities involving hand-eye coordination. A reading surface brightness can be detected automatically, and the light intensity can be adjusted using the IOT-enabled lights (like Alexa, Google, etc.).

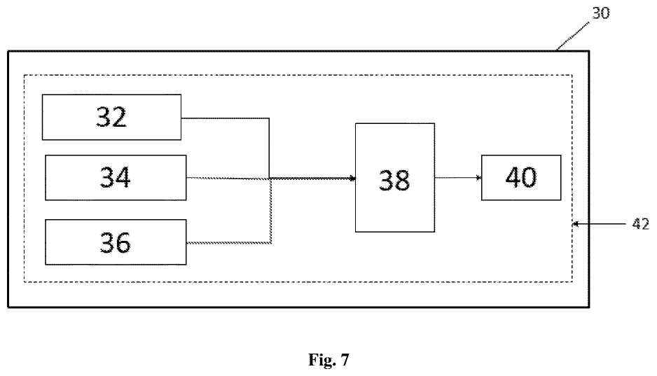

FIG. 7 illustrates a schematic diagram of a device comprising one or more sensors, controller 38, alert unit 40 and power unit 42. The device is comprised of a body 30 that is configured to be attached to an optical wear. The device also includes a controller 38, which comprises a memory and a processor. The controller 38 is designed to process and store data received from the one or more sensors of the device. The device is further equipped with one or more sensors, wherein one or more sensors comprises a gyroscope 32, light sensor 34 and distance sensor 36. The gyroscope 32 is configured to generate an input signal that measures the position of the user's head. The gyroscope 32 is communicatively coupled to the controller 38, allowing them to transmit the input signal to the controller 38 for processing. The controller 38 is configured to compare input signals with one or more predefined threshold values and generate an output signal based on this comparison. An alert unit 40 is also included in the device and is communicatively coupled to the controller 38. The alert unit 40 is configured to generate an alert signal based on the output signal received from the controller 38. This alert signal can be used to notify the user or another device of certain conditions or events. Further, the light sensor 34 is communicatively coupled to the controller 38. The alert unit 40 comprises a buzzer, an alarm or combination thereof. The light sensor 34 is configured to check sufficient illumination on an object while the illumination source provides the necessary light. Furthermore, the distance sensor 36 is communicatively coupled to the controller 38. The distance sensor 36 configured to measure the distance from the device to an object in the user's field of view. The device also includes a power unit 42. The power unit 42 is configured to provide power to the device, allowing it to function and perform its various tasks.

In certain embodiments, the device 10 can be modified to track user eye positions. The device 10 comprises one or more EEG sensors additionally. The one or more EEG sensors can be mounted on the user's head to capture the brain waves. The captured brain waves can be compared with a reference data, to track user eye positions. The reference data can be created by capturing the EEG sensors data while user is instructed to scan a calibration sheet from top to the bottom, during a calibration process. While the user scans the calibration sheet from top to bottom by going through the calibration sheet left to right again and again (like a 2D scanning), the corresponding brain waves are recorded and saved as reference signals for identifying the user's gaze direction and focal point. This reference data is referred to track user eye position, during the real time operation of the device, without using the calibration sheet.

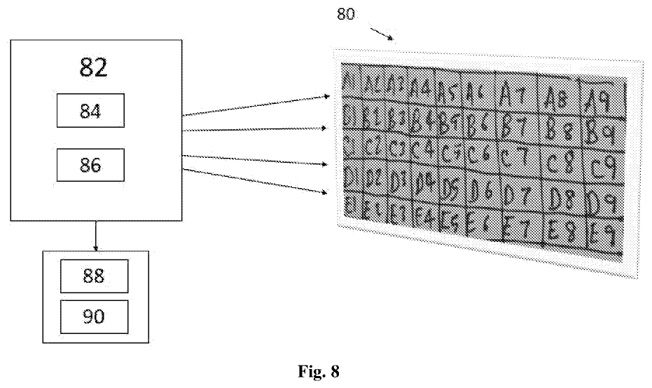

Referring to FIG. 8, the device 90 (corresponding to device 10) also includes a system for tracking the user's eye position post-calibration. The EEG sensors 86 track the user's eye position, which is crucial for applications requiring eye movement tracking, such as augmented reality glasses. This system provides a comprehensive and accurate method of eye position tracking, enhancing the device's 90 functionality and usability. FIG. 8 describes a device 90 for tracking an eye position of a user 82 is illustrated. The device 90 comprises one or more EEG sensors 86 designed to generate one or more input signals. These EEG sensors 86 can be strategically disposed on the user's 82 forehead and mastoid bone to measure the electrical activity in the user's brain.

The device 90 also includes a controller 88, which comprises a memory and a processor. The memory of the controller 88 stores reference data corresponding to the electrical activity in the user's brain. This reference data is generated by reading the brainwave response data from the EEG sensors 86 while the user 82 scans an EEG calibration sheet 80 from top to bottom. The calibration sheet may feature a grid of dots or alphabets.

The device 90 may be programmed using the one or more processors. The controller 88 compares the input signals received from the EEG sensors 86 with the reference data to track the position of the user's eyes 84. This process enables the device 90 to accurately determine the user's eye position and movement.

Further, the device 90 includes a controller 88 comprising a memory and a processor. The memory stores reference data corresponding to the user's brain activity. This reference data is used as a benchmark for comparing the input signals generated by the EEG sensors 86. By comparing the input signals with the reference data, the device 90 can accurately track the user's eye position.

The reference data stored in the memory is generated through a specific process. This process involves reading the brainwave response data generated by the EEG sensors 86 while the user 82 scans an EEG calibration sheet 80 from top to bottom. The EEG calibration sheet 80 can be scanned in various directions, not restricted to top to bottom. Users 82 may be guided to scan from bottom to top or from left to right to comprehensively track the eye's positions. The brainwave response data is then recorded and stored in the memory module as reference data.

In other embodiments, the user 82 can be instructed to focus on any of the grid of dot or any other alphabets. Once, the user 82 focuses on the various grid dots or alphabets or numerals presented on the EEG calibration sheet 80 various brain frequency are generated. The EEG calibration sheet 80 is not limited to the grid of dots or alphabets. Further, to enhance the accuracy of the user's brain activity, the user 82 can be shown different types of pictures to see the user's 82 reaction for a particular image and store the user's 82 response to use as the reference data.

The EEG calibration sheet 80 used in this process may feature a grid of dots or alphabets. This specific design of the calibration sheet aids in the generation of comprehensive reference data. The grid helps facilitate precise measurement of the user's brainwave responses during calibration, contributing to the accuracy of the eye tracking process.

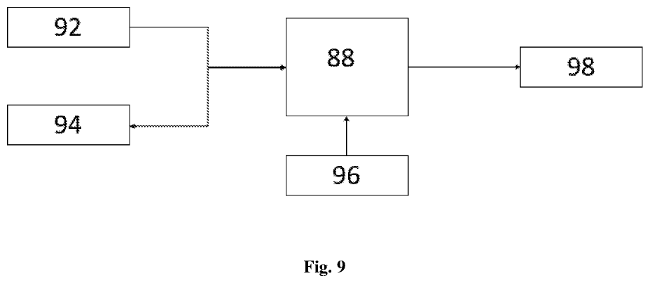

Referring to FIG. 9, the device 90 (as shown in FIG. 8) has been designed to aid dyslexic children in reading. The device 90 includes a movable laser light source 94 that projects light onto reading materials word by word. This feature is designed to help dyslexic children by visually guiding them through each word in their reading material to aid dyslexic children in reading. The device 90 features one or more cameras 92 that are coupled to the controller 88. These cameras 92 are configured to capture an image of the reading material in front of the user 82.

The captured image is then transmitted to a controller 88, which performs an optical character recognition process on the image to extract the words written on the reading material. The device 90 also includes a movable laser light source 94, which projects light onto the reading material.

The device 90 also includes an audio sensor 96 coupled to the controller 88. This sensor is configured to capture an audio signal corresponding to the word pronounced by the dyslexic child. These captured audio signals are processed by the controller 88 to identify the pronounced word within the reading material. The controller 88 then instructs the movable laser light source 94 to move to the next word, visually guiding the child through their reading journey. The moveable laser light source 94 comprises a laser light source, moveable means e.g., electrotechnical motors, and like. The controller 88 instructs or provide control signals to move electrotechnical motors to move or rotate with specific controlled motion steps.

In another embodiments, the controller 88 is also capable of generating an output signal corresponding to the user's mental state. An indicating unit 98 is coupled to the controller 88 and receives this output signal. The indicating unit 98, then generates an alert signal regarding the user's mental state, providing real-time feedback to the user 82 or a caregiver. The present disclosure relates to a device 90 for tracking the eye position of a user 82. The device 90 includes one or more EEG sensors 86 designed to generate input signals. The EEG sensors 86 are configured to measure electrical activity in the user's brain, generating signals that can be used to track the user's eye position. The EEG sensors 86 may be strategically placed on various parts of the user's body, such as the forehead or mastoid bone, to optimally capture this data. The EEG sensor's 86 data can be compared with a second reference data to identify the user's mental state e.g., happy, angry, sad and like.

Further, the device 90 includes a feature that generates an output signal corresponding to the user's mental state. An indicating unit 98, coupled to the controller 88, receives this output signal. The indicating unit 98 then generates an alert signal regarding the user's mental state. This real-time feedback about the user's mental state can provide valuable insights and could be used in various applications such as neurofeedback therapy, cognitive training, or for general monitoring purposes. The indicating unit 98 can be any audio device, visual device or like. In certain embodiments, the device 90 can be connected to a user device, to transfer the status of the user's mental state.

In another embodiments, the present disclosure can be implemented on the user 82 in various scenarios e.g., vehicle driving, wherein the EEG sensors 86 can measure different brainwave frequencies, such as alpha, beta, gamma, delta, and theta changes in these frequencies. The terms alpha, beta, gamma, delta, and theta refer to different frequency ranges of electrical activity in the brain, as measured by EEG sensors. Each of these brain waves is associated with specific states of consciousness and cognitive processes can indicate different emotional states. Alpha waves are also prevalent during the transition from wakefulness to drowsiness and just before falling asleep. When device 90 measure brain frequency of the driver in real time to check the driver's alertness. The measured parameters are transmitted to the controller 88 and the measured parameters are compared with a third reference data comprises reference data associated with specific states of consciousness e, g., drowsy, awake, sleepy or like. If the measured parameter is matched with alpha waves which indication of drowsy or sleepy state, the indicating unit 98 send the alert signal to the driver. The indicating unit 98 can be any audio device, visual device or like. In certain embodiments, the device 90 can be connected to a user device, to transfer the status of the driver's emotional states or state of mind.

In other embodiments, the device 90 could be modified to include additional or alternative solutions. For instance, an array of EEG sensors 86 could be used to generate more comprehensive input signals. Similarly, the design of the EEG calibration sheet 80 could be varied to include different patterns or symbols, enhancing the accuracy of the reference data generated.

The device 90 could also be equipped with advanced image processing capabilities to improve the optical character recognition process. Moreover, the movable laser light source 94 could be augmented with additional features, such as varying light intensity or color, to better support dyslexic children in their reading.

Further, the device 90 can be integrates with a virtual keyboard using Artificial Intelligence (AI). Users visualize words or phrases, and the system, aided by AI, autocorrects and interprets these visualizations in context. This step is essential for creating a mental ‘keyboard’ that responds to thought patterns. This feature enhances the device's usability and accessibility, providing a comprehensive and user-friendly system.

The device 90 also allows users to visualize a spatial canvas where specific focus areas are linked to EEG triggers. This visualization is critical for tasks requiring precision, such as controlling robots or drones. This feature provides a comprehensive and effective system of control, enhancing the device's functionality and usability.

The system also saves user-specific data sets for future use. Subsequent reusing the device 90 require a brief recalibration using predetermined points on the EEG sheet, allowing the system to sync with the user's unique brainwave patterns. This feature ensures the device's accuracy and reliability, providing a personalized and effective system.

The device 90 can be used in a variety of applications, including but not limited to gaming, virtual reality, augmented reality, medical diagnostics, and robotics. The device's comprehensive monitoring system, user-friendly interface, and advanced features make it a versatile and effective solution for a wide range of needs.

The device's features and design provide several advantages. The device provides a comprehensive and accurate system for monitoring the user's head and eye position, enhancing the user's experience in various applications. The device's user-friendly interface and advanced features make it accessible and easy to use. The device's design and features also provide potential benefits to the user's visual health.

The device can also be used in conjunction with other technologies. For example, the device can be integrated with a virtual reality headset to provide a more immersive experience. The device can also be used with a drone or robot to provide a more intuitive and effective control system.

The device's design and features can be further enhanced and expanded. For example, the device can include additional sensors to provide more comprehensive monitoring. The device can also include more advanced AI features to provide a more personalized and effective system. The device's design can also be made more ergonomic to enhance the user's comfort and ease of use.

In some embodiments, the device 90 may be connected to one or more user devices using a software application. In such embodiments, the software application may be designed to control one or more functionalities of the device. The application may be installed in a user device 90. In an embodiment, the user device may be connected to a server using a communication network. Different parameters selected or entered by the user through the user device may be transmitted to the device 90 via the server. In another embodiment, the user device may be connected to using near-field communication, or Bluetooth. In such embodiments, values of such parameters may be received by the one or more processors of the device 90 so as to control the functioning of different parts such as the one or more sensors or the at least indicating unit.

It is submitted that even though the use of the device throughout the description is illustrated with optical wear of the user, the device may be used with other devices as well. For instance, the device may be attached to headbands, antiglare glasses, virtual reality (VR) glasses, headsets, or the like. Variations of this disclosure can be integrated into VR headsets and data collected can be used to design VR headsets.

The foregoing discussion of the present disclosure has been presented for purposes of illustration and description. It is not intended to limit the present disclosure to the form or forms disclosed herein. In the foregoing Detailed Description, for example, various features of the present disclosure are grouped together in one or more embodiments, configurations, or aspects for the purpose of streamlining the disclosure. The features of the embodiments, configurations, or aspects may be combined in alternative embodiments, configurations, or aspects other than those discussed above. This method of disclosure is not to be interpreted as reflecting an intention the present disclosure requires more features than are expressly recited in each claim.

Moreover, though the description of the present disclosure has included description of one or more embodiments, configurations, or aspects and certain variations and modifications, other variations, combinations, and modifications are within the scope of the present disclosure, e.g., as may be within the skill and knowledge of those in the art, after understanding the present disclosure, it is intended to obtain rights which include alternative embodiments, configurations, or aspects to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. A device (10) for monitoring a position of head of a user, the
device (10) comprising:
a body (30) configured to be attached to an optical wear (1);
a controller (38), wherein the controller (38) comprises a memory and a processor;
one or more sensors, wherein the one or more sensors are configured to generate one or more input signals to measure the position of the user's head;
an alert unit (40) communicatively coupled to the controller (38); and
a power unit (42);
wherein the one or more sensors communicatively coupled to the controller (38) to transmit the one or more input signals to the controller (38);
wherein the controller (38) is configured to compare the one or more input signals with corresponding one or more predefined threshold values and generate an output signal;
wherein the alert unit (40) is configured to generate an alert signal based on the output signal received from the controller (38);
wherein the one or more sensors comprises a gyroscope (32), wherein the gyroscope (32) is configured to measure an angle of inclination of the user's head with respect to X, Y, and Z planes and the one or more predefined threshold values comprises a threshold angle of inclination of the user's head.

2. The device (10) claimed in claim 1, wherein the one or more sensors further comprises a light sensor (34), wherein the light sensor (34) is configured to check sufficient illumination on an object in the user's field of view; and
wherein the one or more predefined threshold values further comprises a threshold illumination value.

3. The device (10) claimed in claim 1,
wherein the one or more sensors further comprises a distance sensor (36) configured to measure the distance from the device to an object in the user's field of view;
wherein the one or more predefined threshold value further comprises a threshold distance value; and
wherein distance sensor (36) comprises a lidar sensor.

4. The device (10) claimed in claim 1, the controller (38) transmits the output signal to the alert unit (40) upon detecting the input signal received from the one or more sensors is more than the one or more corresponding predefined threshold values.

5. The device (10) claimed in claim 1, further comprises an illumination source positioned such as to face the user's optic nerve, wherein the illumination source is configured to provide a blue light and a red light to stimulate the optic nerve of the user to improve eyesight.

6. The device (10) claimed in claim 1, wherein the alert unit (40) comprises a buzzer, an alarm or combination thereof.

7. The device (10) claimed in claim 1, wherein the power unit (42) comprises a rechargeable battery.

8. A device (90) for tracking an eye position of a user, the device comprising:
one or more EEG sensors (86) to generate one or more input signals;
a controller (88), the controller (88) comprises a memory and a processor;
wherein the memory comprises a reference data corresponding to an electrical activity in the user's brain; and
wherein the controller (88) is configured to compares the one or more input signals received from the one or more EEG sensors (86) with the reference data to track the position of the user's eyes.

9. The device (90) claimed in claim 8, wherein a process of generating the reference data comprises:
reading a brainwave response data generated from the one or more EEG sensors (86) disposed on a forehead and a mastoid bone of the user to measure the electrical activity in the user's brain using the controller (88), while user is to scan an EEG calibration sheet (80) from top to bottom; and
storing the measured brainwave response data in the memory as the reference data by the controller (88).

10. The device (90) claimed in claim 9, wherein the EEG calibration sheet (80) features a grid of dots or alphabets.

11. The device (90) claimed in claim 8, additionally designed to project a movable laser light, word by word on a reading material positioned in front of a dyslexic children to aid in reading, further comprises:
one or more cameras (92), the one or more cameras (92) coupled to the controller (88), wherein the one or more cameras (92) are configured to capture an image of the reading material;
wherein the captured image is transmitted to the controller (88) to perform an optical character recognition process on the image to extract words written on a reading material;
a movable laser light source (94);
an audio sensor (96) coupled to the controller (88), the audio sensor (96) is configured to capture an audio signal corresponding to a word pronounced by the dyslexic children; and
the controller (88) processes the audio signal to locate the pronounced word in the reading material and instruct the movable laser light source (94) to move and project light to a next word to the pronounced word on the reading material.

12. The device (90) claimed in claim 8,
wherein the controller (88) generates an output signal corresponding to the user's mental state; and
an indicating unit (98) coupled to the controller (88), wherein the indicating unit (98) receive the output signal from the controller (88) to generate an alert signal regarding the user's mental state.
